(19) United States
(12) Patent Application Publication
(10) Pub. No.: US 2003/0032999 A1
(43) Pub. Date: Feb. 13, 2003

(54) Balloon stent assembly system and method

(75) Inventor: Mark Huang, Santa Rosa, CA (US)

Correspondence Address: MEDTRONIC AVE, INC., 3576 UNOCAL PLACE, SANTA ROSA, CA, 95403, US

(73) Assignee: Medtronic AVE, Inc.

(21) Appl. No.: 09/924,320

(22) Filed: Aug. 7, 2001

(51) Int. Cl. A61F 2/06

(52) U.S. Cl. 623/1.11

(57) ABSTRACT

A balloon stent assembly system and method for retaining the stent on the same. The system includes a balloon including an outer layer portion; and a stent including a mesh portion disposed on the balloon, the mesh portion covering at least 55 percent of the outer layer portion. The outer layer portion flows into gaps formed in the stent when the balloon stent assembly is heated to a predetermined temperature, and retains the stent on the balloon during intravascular movement. The method of retaining a stent on a balloon includes: mounting the stent onto the balloon, sheathing the mounted stent and balloon, heating the balloon, and flowing an outer layer of the balloon into the gaps formed in the stent while an inner layer of the balloon does not flow.

### TECHNICAL FIELD OF THE INVENTION

The present invention relates to medical implant devices. Specifically, the invention relates to a balloon encapsulated by an expandable stent for intravascular deployment.

### BACKGROUND OF THE INVENTION

Balloon catheters are used in a variety of medical therapeutic applications including intravascular angioplasty. For example, a balloon catheter device is inflated during PTCA (percutaneous transluminal coronary angioplasty) to dilate a stenotic blood vessel. The stenosis may be the result of a lesion such as a plaque or thrombus. After inflation, the pressurized balloon exerts a compressive force on the lesion thereby increasing the inner diameter of the affected vessel. The increased interior vessel diameter facilitates improved blood flow. Soon after the procedure, however, a significant proportion of treated vessels re-narrow.

To prevent restenosis, short flexible cylinders, or stents, constructed of metal or various polymers are implanted within the vessel to maintain lumen size. The stent acts as a scaffold to support the lumen in an open position. Various configurations of stents include a cylindrical tube defined by a mesh, interconnected stents or like segments. Some exemplary stents are disclosed in U.S. Pat. No. 5,292,331 to Boneau, U.S. Pat. No. 6,090,127 to Globerman, U.S. Pat. No. 5,133,732 to Wiktor, U.S. Pat. No. 4,739,762 to Palmaz and U.S. Pat. No. 5,421,955 to Lau. Balloon-expandable stents are mounted on a collapsed balloon at a diameter smaller than when deployed. During the procedure, the balloon stent catheter is advanced through a network of tortuous blood vessels. Furthermore, the balloon stent catheter also may encounter narrowed lumens or lumens that are obstructed. Once at the desired site, the balloon is inflated and expands the stent to a final diameter. After deployment, the stent remains in the vessel and the balloon catheter is removed.

While the balloon stent catheter is moved longitudinally through the network of vessels, position of the stent should be maintained. The stent may become dislodged off the balloon or if it is shifted on the balloon, it may not expand fully along its length. Current strategies for retaining the stent on the balloon include: plastically deforming the stent so that it is crimped onto the balloon; increasing the friction forces between the stent and balloon by modifying the balloon through heat, pressure, or chemical or adhesive means; adding retainers that physically prevent the stent movement; or combinations thereof.

U.S. Pat. No. 4,950,227 to Savin discloses a strategy for stent retention that utilizes end caps mounted on the catheter. The end caps are adapted to temporarily engage the ends of the stent while permitting the stent ends to release when the stent is expanded.

U.S. Pat. Nos. 5,836,965 and 6,159,229 issued Dec. 12, 2000 to Jendersee et al. discloses a strategy for stent retention utilizing a heating process to deform the balloon about the stent while the balloon is heated and preferably pressurized. The balloon expands around and within gaps of the stent causing it to adhere. The balloon continues to adhere as it is cooled and its shape is set. Furthermore, retainers may be placed at the distal and/or proximal ends of the stent.

The U.S. Pat. No. 5,976,181 issued Nov. 2, 1999 to Whelan et al. discloses a strategy for stent retention utilizing a chemical process to deform the balloon. The balloon is sheathed and pressurized followed by the addition of solvent. The process produces radial projections in the balloon surface that are forced around the ends and within the gaps of the stent. The balloon is depressurized and maintains a permanent shape that interlocks with the stent.

The U.S. Pat. No. 6,066,156 issued May 23, 2000 to Yan discloses a strategy for stent retention utilizing a temperature activated releasable adhesive. The tacky adhesive coated balloon increases stent retention. The adhesive becomes non-tacky at a transformation temperature just above that of human blood. After positioning the stent at the desired site, a warming solution is introduced to transform the adhesive. The adhesive becomes non-tacky and releases the stent.

The U.S. Pat. No. 6,110,180 issued Aug. 29, 2000 to Foreman et al. discloses a strategy for stent retention utilizing an expandable member having outwardly extending protrusions. The protrusions are formed by applying dots of an adhesive material on the outer surface of the expandable member. Alternatively, the protrusions may be integrally formed with the balloon. The stent is crimped onto the expandable member such that the protrusions extend into the gaps of the stent. After deployment, the protrusions are retracted from the gaps thereby releasing the stent.

The disclosed and other strategies may provide adequate stent retention for a number of older stent designs. A current trend for stent design, however, calls for an increasing density of mesh struts or segments forming the scaffolding to enhance mechanical strength, reduce failure and increase the stent to artery ratio. The newer stents have smaller interstices, or gaps, between the mesh struts or segments. As a result, many of the existing strategies cannot provide sufficient stent retention required during intravascular maneuvers. For example, balloons deformed by a heat/pressure process may not extend sufficiently into the smaller gaps to adequately secure the stent on the balloon.

When looking at ways to improve stent retention, one must not compromise the design considerations of longitudinal flexibility and low profile of the stent for deliverability. Other limitations of current stent retention strategies include: mechanical retainers and permanent sheaths that may increase unit profile and cost; crimping which may permanently deform the stent and hinder deployment; tacky adhesives that may complicate catheter advancement through a vessel; and multiple layers that may increase balloon rigidity and cost. Therefore, it would be desirable to achieve a balloon-stent assembly that is compatible with newer stent designs and to overcome the aforementioned and other disadvantages.

### SUMMARY OF THE INVENTION

One aspect of the invention provides stent delivery system comprising a balloon, including an outer layer portion, and a stent. The stent covers at least 55 percent of the outer layer portion. The outer layer portion flows into gaps formed in the stent when the balloon delivery system is heated to a predetermined temperature securing the stent on the balloon. The predetermined temperature may range from about 50 to 70 degrees Celsius. The balloon comprises at least one outer layer and at least one inner layer, the outer layer portion comprising the outer layer. The outer and inner layers may comprise a co-extruded laminate. Furthermore, the outer layer may comprise a tie layer and/or a functionalized material. The functionalized material is not tacky at temperatures below the predetermined temperature and may consist of polyethylene.

Another aspect of the invention provides a balloon stent delivery system comprising a balloon including at least one non-tacky outer layer and at least one inner layer, and a stent disposed on the outer layer. When the balloon is heated at a predetermined temperature an outer layer portion flows into gaps formed in the stent while the inner layer does not flow. The outer layer comprises a first material and the inner layer comprises a second material different from the first material. The stent may cover at least 55 percent of the outer layer and the balloon may provide at least 200 gf (gram force) of a stent retention force. The stent may cover at least 70 percent of the outer layer and the balloon may provide at least 300 gf of a stent retention force. The stent may cover at least 90 percent of the outer layer and the balloon may provide at least 90 gf of a stent retention force.

Yet another aspect of the invention provides for a method of retaining a stent on a balloon comprising: mounting the stent onto the balloon, the stent including gaps formed therein, sheathing the mounted stent and balloon, heating the balloon, and flowing an outer layer of the balloon into the gaps formed in the stent while an inner layer of the balloon does not flow. The balloon may be pressurized. The outer layer may flow into a predetermined or a random arrangement of gaps.

The foregoing and other features and advantages of the invention will become further apparent from the following detailed description of the presently preferred embodiments, read in conjunction with the accompanying drawings. The detailed description and drawings are merely illustrative of the invention rather than limiting, the scope of the invention being defined by the appended claims and equivalents thereof.

### BRIEF DESCRIPTION OF THE DRAWINGS

FIG. 1 is a sectional view of one embodiment of a balloon stent assembly system disposed on a catheter according to the present invention;

FIG. 2 is a sectional view of the balloon stent assembly system of FIG. 1 after an outer layer portion has flowed into stent gaps;

FIG. 3 is a cross-sectional view of the balloon stent assembly system of FIG. 2; and

FIG. 4 is a sectional view of a sheathed balloon stent assembly of the present invention.

### DETAILED DESCRIPTION OF THE PRESENTLY PREFERRED EMBODIMENT

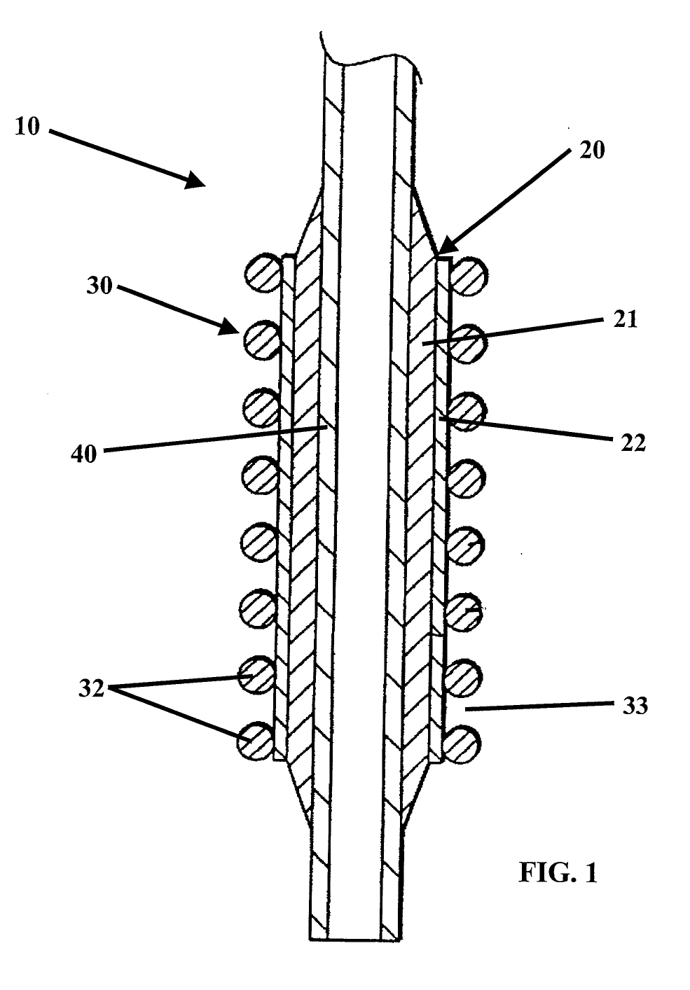

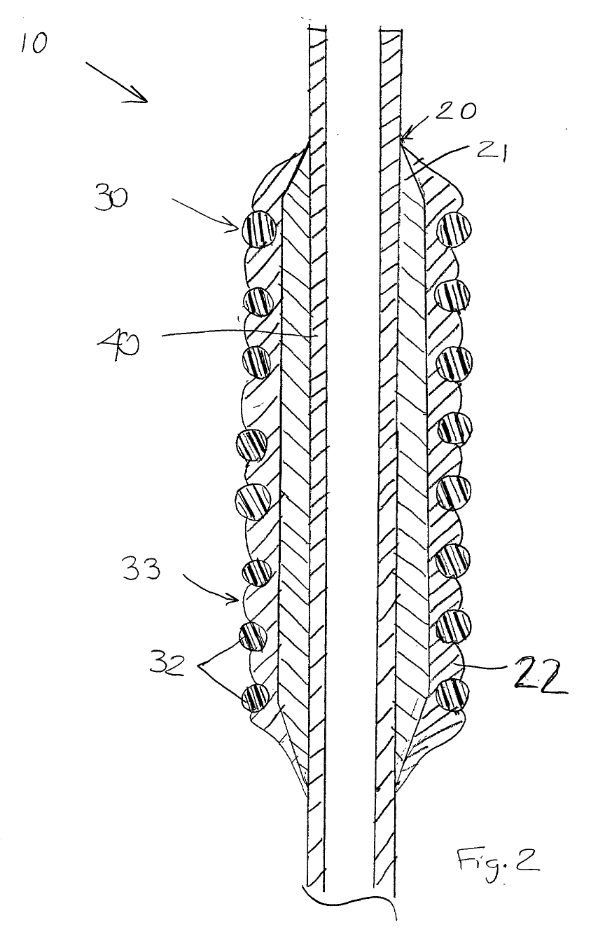

Referring to the drawings, illustrated in FIGS. 1 and 2 is one embodiment of a balloon stent assembly 10 disposed on a catheter 40 in accordance with the present invention. The balloon stent assembly 10 comprises a balloon 20, including an outer layer portion 22, and a stent 30, disposed on the balloon 20. The stent 30 covers at least 55 percent of the outer layer portion 22. The outer layer portion 22 is defined as the part of the balloon 20 or its components that are in operable contact with the stent 30, and the members 32, and gaps 33 that comprise the stent 30.

The balloon 20 is shown in a collapsed position and may be inflated during stent 30 deployment. The stent 30 comprises a lattice configuration of members 32, fabricated from metal or other structural material, and gaps 33 formed between the members 32. The stent 30 may additionally be crimped onto the balloon to enhance retention. The balloon stent assembly 10 is typically delivered to a desired site utilizing a standard guidewire for negotiation of vessel pathways.

The balloon 20 comprises at least one non-tacky outer layer 22 and at least one inner layer 21. The outer 22 and inner layers 21 may comprise a co-extruded laminate. Furthermore, the outer layer may comprise a tie layer and/or a functionalized material. An example of a co-extruded trilayer laminate including a tie layer is disclosed in U.S. Pat. No. 6,165,166 issued to Samuelson et al. and is incorporated herein in its entirety by reference thereto. As shown in FIGS. 1 and 2, the outer layer 22 is disposed on a limited area of the inner layer 21. It can be appreciated that the outer layer 22 may cover varying proportions and configurations of the inner layer 21 to achieve desirable stent retention.

The inner layer 21 may be fabricated from a variety of biocompatible compliant or non-compliant materials or blends including nylon-12 and Selar®. The functionalized material and potentially the tie layer are not tacky at temperatures below the predetermined temperature and may comprise a polyethylene such as Bynel® or Plexar®. Alternatively, the inner 21 and outer layers 22 may be comprised of a number of material(s) or layer(s) that adequately provides both support and stent retention.

When the balloon stent assembly is heated to a predetermined temperature, the outer layer portion 22 flows into gaps 33 formed in the stent 30. The predetermined temperature may range from about 50 to 70 degrees Celsius. The predetermined temperature will vary based on characteristics of the functionalized material. For example, a temperature of 100 degrees Celsius may be required to flow a functionalized material adequately into the gaps 33.

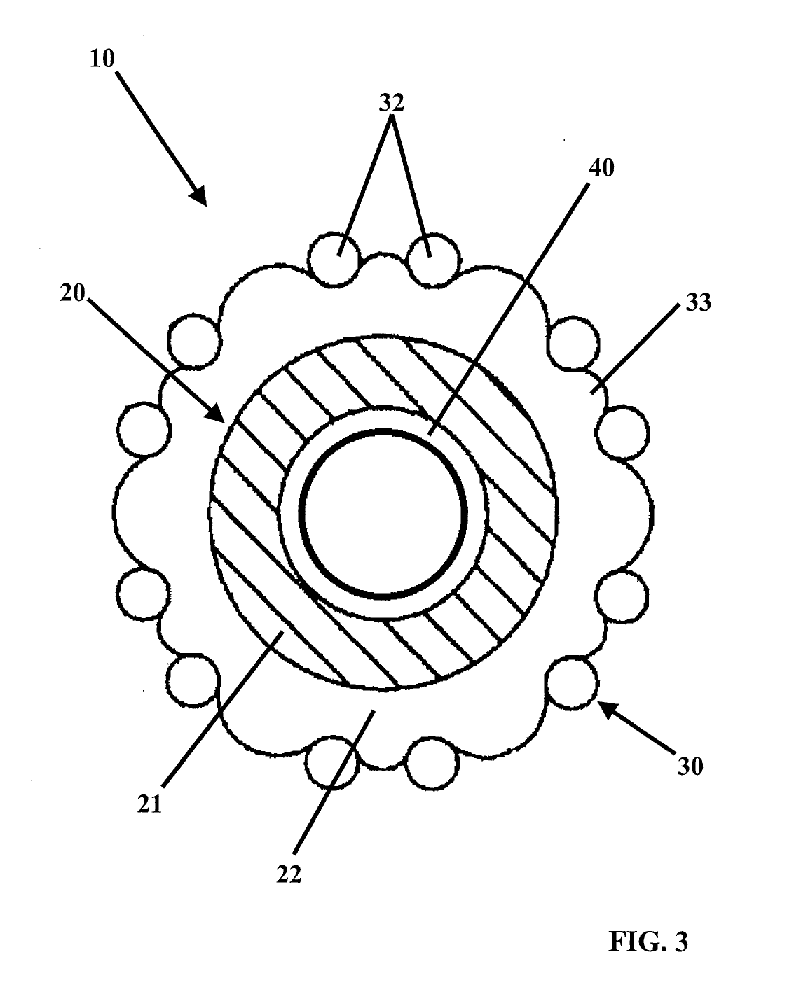

FIGS. 2 and 3 are representations of one embodiment after the outer layer portion 22 has flowed into the gaps 33. The flowed outer layer portion 22 retains the stent 30 on the balloon 20 during intravascular movement. The relatively low viscosity of the outer layer portion 22 allows for flow into smaller gaps 33 and, thus, enhances stent 30 retention. In addition, the outer layer portion 22 is adaptably capable of flowing into a range of gap 33 sizes present within the stent 30.

In another embodiment, an outer layer portion 22 flows into gaps 33 while an inner layer 21 does not flow when heated to the predetermined temperature. Furthermore, the outer layer 22 comprises a first material and the inner layer 21 comprises a second material different from the first material.

Yet another embodiment includes a method of retaining a stent 30 on a balloon 20 comprising: mounting the stent 30 onto the balloon 20, the stent 30 including gaps 33 formed among members 32 that covers at least 55 percent of the balloon, sheathing the mounted stent 30 and balloon 20, heating the balloon 20, and flowing an outer layer 22 of the balloon 20 into the gaps 33 formed in the stent 30 while an inner layer 21 of the balloon 20 does not flow. The balloon 20 may be pressurized to facilitate the flow of the outer layer 22 into the gaps 33. This heat set procedure may be similar to the processes disclosed by U.S. Pat. No. 6,032,092 issued to Shin and U.S. Pat. No. 6,159,229 issued to Jendersee at al. and are incorporated herein in their entirety by reference thereto.

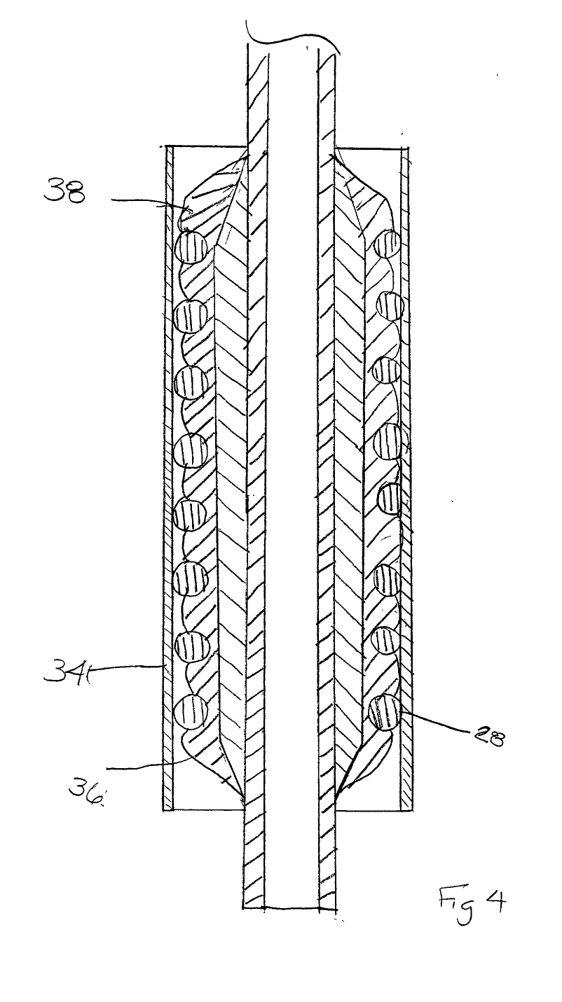

In such a procedure, a tube made of a sufficiently rigid material such as metal, plastic, or the like is placed around the balloon stent assembly 10 to maintain a limited inflation size. The sheathed balloon stent assembly (shown in FIG. 4) may then be pressurized with an inflation pressure, for example, in the range of approximately ten to twenty pounds per square inch. The sheathed assembly is then heated to the predetermined temperature for a time sufficient to allow adequate flow of the outer layer portion 22 into the gaps 33. The balloon 20 portion or outer layer portion 22 may flow into a predetermined or a random arrangement of gaps 33 depending on the stent 30 design and outer layer 22 composition. Furthermore, the stent assembly may include a distal retainer 36 and/or a proximal retainer 38 to further secure the stent to the balloon. The retainers also create a transition between the balloon and stent area of delivery device and the distal and proximal surfaces of the delivery device of the encapsulated stent assembly. The retainers may be formed by the balloon itself during the encapsulation process, with the configuration of the formed retainers determined by the dimensions of the spaces between the sheath 34 and the stent members 32. Upon completing the flowing step, the sheathed balloon stent assembly is allowed to cool to room temperature and the tube sheath is removed. The functionalized material is not tacky at this point thereby preventing any exposed portion from adhering to vessel surfaces during medical procedures.

The stent 30 can be of a typical stent design, including wire and tube designs, and may expand during deployment. The present invention is compatible with older stent designs such as those disclosed in, for example, U.S. Pat. No. 5,649,952 issued to Lam and U.S. Pat. No. 5,514,154 issued to Lau et al. In addition, improved stent retention is demonstrated for newer stent designs covering in excess of 55 percent of the outer layer portion 22.

The current trend for stent design calls for an increasing density of members 32 to enhance mechanical strength and reduce failure. The novel stents have smaller interstices, or gaps 33, between the members 32. The balloon 20 area covered by the stent 30 generally influences stent retention force. The retention force typically diminishes, particularly with current retention technology, as the gaps 33 decrease in size. Balloon stent assemblies 10 made in accordance with the present invention, however, provides a greater retention force when compared to the existing designs.

Experiments using balloon stent assemblies 10 made in accordance with the present invention reveal a stent retention force of 200 gf (gram force) or greater when the stent 30 covers about 55 to 70 percent of the outer layer portion 22. Stents covering about 70 to 90 percent provide at least 300 gf, and stents covering about 90 percent and greater provide at least 90 gf of retention force. Therefore, the present invention provides a superior stent retention force and may reduce the chance of stent slippage or loss during intravascular movement.

For illustrative purposes, the following table gives approximate stent retention force for several newer stent designs. The table outlines: the outer portion coverage percentage, retention force using existing balloon stent retention technology (retention A), retention force using a balloon stent assembly made in accordance with the present invention (retention B), and percent difference of retention A to B (% Δ).

TABLE I

| Stent | Covered Area (%) | Retention A (gf) | Retention B (gf) | % Δ |
|---|---|---|---|---|
| 1 | 58 | 200 | 220 | +10 |
| 2 | 65 | 72 | 204 | +183 |
| 3 | 72 | 159 | 399 | +151 |
| 4 | 72 | 136 | 318 | +134 |
| 5 | 91 | 40 | 94 | +135 |

The invention and its detailed embodiments are described as applied for use in coronary arteries, i.e. during PTCA. Those skilled in the art will appreciate that the invention may be applied to devices for use in other body lumens as well, such as peripheral arteries and veins. Also, the invention is described with respect to the balloon stent assembly 10 disposed on a portion of a catheter 40. The invention may be mounted on any device capable of delivering the assembly to a required site.

While the embodiments of the invention disclosed herein are presently considered to be preferred, various changes and modifications can be made without departing from the spirit and scope of the invention. The scope of the invention is indicated in the appended claims, and all changes that come within the meaning and range of equivalents are intended to be embraced therein.

1. A balloon stent assembly system comprising:
a balloon including an outer layer portion; and
a stent disposed on the balloon, the stent covering at least 55 percent of the outer layer portion;
wherein the outer layer portion flows into gaps formed in the stent when the balloon stent assembly is heated to a predetermined temperature, and retains the stent on the balloon during intravascular movement.

2. The system of claim 1 wherein the predetermined temperature comprises a temperature range of about 50 to 70 degrees Celsius.

3. The system of claim 1 wherein the balloon comprises at least one outer layer and at least one inner layer, the outer layer portion comprising the outer layer.

4. The system of claim 3 wherein the outer layer and the inner layer comprise a co-extruded laminate.

5. The system of claim 3 wherein the outer layer comprises a tie layer material.

6. The system of claim 3 wherein the outer layer comprises a functionalized material.

7. The system of claim 6 wherein the functionalized material comprises at least one material selected from a group consisting of: polyethylene, ethylene-vinyl-acetate, acrylate, Bynel®, and Plexar®.

8. The system of claim 6 wherein the functionalized material is not tacky at temperatures below the predetermined temperature.

9. A balloon stent assembly system comprising;
a balloon including at least one non-tacky outer layer and at least one inner layer; and
a stent disposed on an outer layer portion;
wherein when the balloon is heated at a predetermined temperature an outer layer portion flows into gaps formed in the stent while the inner layer does not flow.

10. The system of claim 9 wherein the stent covers at least 55 percent of the outer layer portion.

11. The system of claim 10 wherein the balloon provides at least 200 gram force of a stent retention force.

12. The system of claim 9 wherein the stent covers at least 70 percent of the outer layer portion.

13. The system of claim 12 wherein the balloon provides at least 300 gram force of a stent retention force.

14. The system of claim 9 wherein the stent covers at least 90 percent of the outer layer portion.

15. The system of claim 14 wherein the balloon provides at least 90 gram force of a stent retention force.

16. The system of claim 9 wherein the predetermined temperature comprises a temperatures range of about 50 to 70 degrees Celsius.

17. The system of claim 9 wherein the outer layer and the inner layer comprise a co-extruded laminate.

18. The system of claim 9 wherein the outer layer comprises a tie layer material.

19. The system of claim 9 wherein the outer layer comprises a first material and the inner layer comprises a second material different from the first material.

20. The system of claim 19 wherein the first material comprises polyethylene.

21. The system of claim 19 wherein the first material is not tacky at temperatures below the predetermined temperature.

22. A method of retaining a stent on a balloon comprising:
mounting the stent onto the balloon, the stent including gaps, the stent covering at least 55 percent of the balloon;
sheathing the mounted stent and balloon;
heating the balloon; and
flowing an outer layer of the balloon into the gaps formed in the stent while an inner layer of the balloon does not flow.

23. The method of claim 22 wherein heating the balloon comprises elevating the balloon temperature to a temperature of about 50 to 70 degrees Celsius.

24. The method of claim 22 wherein the outer layer flows into a predetermined gap arrangement.

25. The method of claim 22 wherein the outer layer flows into a random gap arrangement.

26. The method of claim 22 further comprising pressurizing the balloon.

27. A balloon comprising:
a outer layer means for flowing into gaps formed between struts on a stent; and
an inner layer means for supporting the outer layer means.
